(19) United States
(12) Patent Application Publication
(10) Pub. No.: US 2025/0249271 A1
(43) Pub. Date: Aug. 7, 2025

(54) SYSTEM FOR ADMINISTERING TRANSCRANIAL MAGNETIC STIMULATION

(71) Applicants: Adam Bied, Reading, MA (US); Susan Njuguna, Reading, MA (US)

(72) Inventors: Adam Bied, Reading, MA (US); Susan Njuguna, Reading, MA (US)

(21) Appl. No.: 18/430,842

(22) Filed: Feb. 2, 2024

(51) Int. Cl. A61N 2/00; A61N 2/02

(52) U.S. Cl. CPC A61N 2/006; A61N 2/02

(57) ABSTRACT

A system for administering transcranial magnetic stimulation in which two or more coils are applied to the patient's body. The coils each may emit a magnetic field which when positioned in one or more configurations result in field overlapping. Magnetic field overlap achieves therapeutic effects otherwise difficult, or impossible, to achieve from traditional single coil TMS devices.

### BACKGROUND OF THE INVENTION

### Field of the Invention

The present invention generally relates to a system for administering transcranial magnetic stimulation (TMS), and more particularly to a system for administering TMS employing more than a single coil.

### Description of the Related Art

Transcranial magnetic stimulation (TMS) has shown diagnostic and therapeutic potential for a diversity of central nervous system (CNS) disease states, notably in the fields of neurology and mental health. Despite the success and potential of this treatment, some proportion of patients are intolerant, or poorly tolerant, of TMS, have medical contraindications to its use, or experience brain disease for which the anatomic target is difficult to reach. A potential contraindication to conventional TMS treatments includes the presence, or potential presence, of any ferromagnetic material in close proximity to the target of treatment or in a physical position orthogonal to the path between the coil and the treatment target. Another issue pertains to localized discomfort experienced when administering TMS. Tissues unrelated to treatment, but in the path of the magnetic field, can also be subjected to magnetic field during treatment, inducing localized discomfort. Conventional TMS coils also experience limits to the depth with which they can exert their therapeutic effects, are sometimes easier to recognize than sham, placebo, counterparts in research or quality improvement activities and are vulnerable to wear from repeated use.

Further, it is known that TMS employs electromagnetic induction to create an electric current across the scalp and skull. Conventional TMS devices employ an enclosed coil of wire applied in close proximity to the skull, which, when activated, produces varying magnetic fields oriented orthogonally to the plane of the coil. Changing magnetic fields induces an electric current in brain tissue which in turn activates neurons or other cells. The magnetic field of conventional TMS devices is similar to that of a Magnetic Resonance Imager (MRI). A pulse of a conventional TMS device generally reaches no more than 5 centimeters into the brain unless a modified coil and/or technique is used for deeper stimulation. Deep TMS is a currently employed variant of TMS which can reach up to 6 centimeters into the brain to stimulate deeper layers of the motor cortex. There may be instances where a patient may benefit from TMS pulses that can reach even deeper than 6 centimeters into the brain or in which conventional, single coil, deep TMS may be capable of reaching the desired target, but which also induces intolerability, insufficient treatment response due to poor field strength, or involvement of areas of the body harboring ferromagnetic materials.

A device which enabled stimulation of an anatomic target without also involving other brain regions, including those in an orthogonal path from the skull surface, would hence be more tolerated, allow for use of the treatment in circumstances in which a ferromagnetic material is in the path of magnetic field or proximity to the target, permit use in circumstances where stimulation of a brain region more proximal to the target is best not done, and allow treatment at depths greater than 5 cm from the skull surface.

Therefore, a TMS system is required that can overcome the challenges associated with the conventional TMS devices.

### SUMMARY OF THE INVENTION

The following presents a simplified summary of the present disclosure in a simplified form as a prelude to the more detailed description that is presented herein.

In accordance with embodiments of the invention, there is provided a system for administering transcranial magnetic stimulation (TMS). The system may include a first coil configured to be adhered to a patient's body part. The first coil may emit a first magnetic field. The system may further include a second coil configured to be adhered to the patient's body part simultaneously with the first coil. The second coil may emit a second magnetic field. The first coil and the second coil may be adhered to different portions of the patient's body part. The first magnetic field and the second magnetic field may overlap within the patient's body part, creating a zone within which the combined field strength exceeds that possible with a single coil alone. The principle so described may further be potentiated by use of three or more coils, configured to induce fields overlapping with one another.

Applicant has surprisingly discovered that use of multiple magnetic field sources allow a provider to use TMS for treatment of patients having counterindications to prior Repetitive Transcranial Magnetic Stimulation (rTMS) treatment, for example due to the presence of a pacemaker, skull plate, shrapnel, or other metal materials in the patient's skull, to induce TMS's treatment effects in areas of the brain where the required field strength impacting the target would otherwise be insufficient, or to administer the required TMS magnetic field strength at the target without also inducing pronounced involvement of more superficial, non-target tissue.

In some aspects, the patient's body part may be the patient's brain. In other aspects, the patient's body part may be the patient's brainstem, spinal cord, or peripheral nervous system.

In further embodiments, the system may include a third coil configured to be adhered to the patient's body part simultaneously with the first coil and the second coil. The third coil may emit a third magnetic field. In some aspects, the first magnetic field, the second magnetic field and the third magnetic field may overlap within the patient's body part. Furthermore, the first coil, the second coil and the third coil may be adhered to different portions of the patient's body part. The same principle could be employed with a fourth, fifth, or any number of additional coils.

The present disclosure describes a novel system for administering transcranial magnetic stimulation (TMS). The system employs two or more coils in a configuration by which the magnetic field extends in space to a common area of overlap coinciding with the tissue target of interest. The system enables greater magnetic field strength than experienced at any other more superficial site. The system provides a plurality of advantages, e.g., reduces the potential for involvement of other non-target tissues or ferromagnetic materials, including tissues/materials which are more superficial on the orthogonal plane from the coil to the target site. The system further offers better conceal treatment versus sham or placebo TMS, and enables more powerful, or deeper penetrating, magnetic field, particularly to the depths exceeding the present maximum of 6 centimeters. The system further reduces the demand, and maintenance needs, of TMS coils by exerting less pronounced electrical currents on any given coil.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which:

FIG. 1 depicts a side elevation view of a system for administering transcranial magnetic stimulation (TMS) in accordance with embodiments of the invention.

FIG. 2 depicts a front elevation view of the system of FIG. 1 in accordance with embodiments of the invention.

### DETAILED DESCRIPTION OF INVENTION

For a further understanding of the nature and function of the embodiments, reference should be made to the following detailed description. Detailed descriptions of the embodiments are provided herein, as well as the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure, or manner. It should be understood that the devices, materials, methods, procedures, and techniques described herein are presently representative of various embodiments. Other embodiments of the disclosure will readily suggest themselves to such skilled persons having the benefit of this disclosure.

Reference will now be made in detail to the present preferred embodiments of the invention, examples of which are illustrated in the accompanying drawings. Wherever possible, the same reference numerals are used in the drawings and the description to refer to the same or like parts.

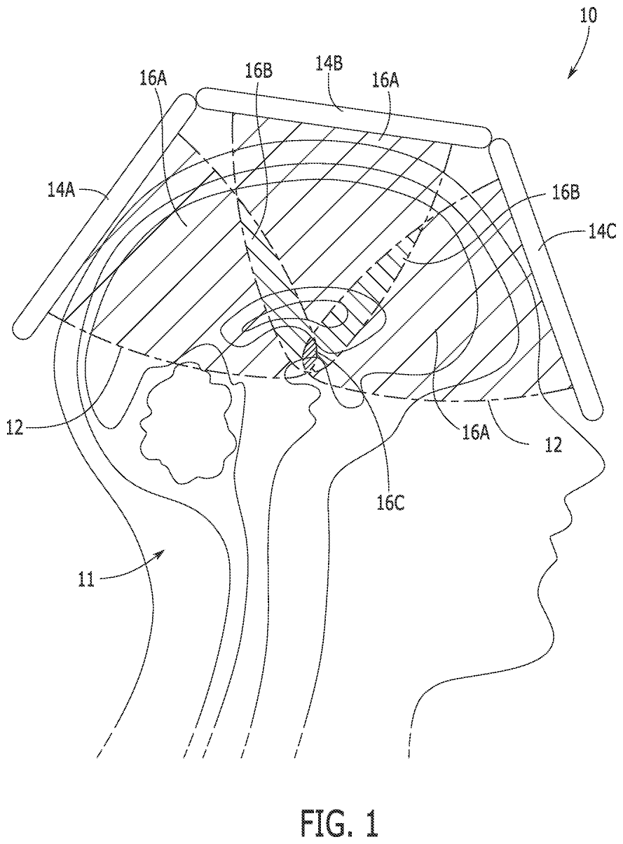

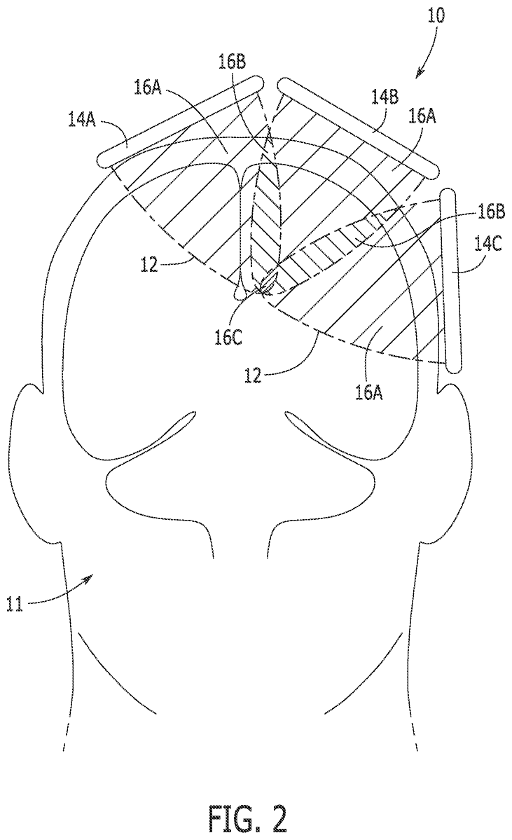

FIG. 1 depicts a side elevation view of a system 10 for administering transcranial magnetic stimulation (TMS) in accordance with embodiments of the invention. FIG. 1 will be described in conjunction with FIG. 2, which depicts a front elevation view of the system 10 in an alternate configuration. The system 10 may be adhered to or placed on a head of a patient 11. The system 10 may include a plurality of coils, e.g., a first coil 14A, a second coil 14B, and a third coil 14C, which may emit coil fields 12 into the patient's head/brain. The fields 12 emitted by the first coil 14A, the second coil 14B, and the third coil 14C may enable transcranial magnetic stimulation on the patient 11 at strengths and/or locations that have not been achieved to date. In an exemplary aspect, the fields emitted by the first coil 14A, the second coil 14B, and the third coil 14C may overlap with each other to provide stronger (and more powerful) magnetic fields to the patient's brain. Each single coil field (i.e., the field which is not overlapped with other fields) is shown as a coil field 16A in FIGS. 1 and 2, a double overlaid coil field (i.e., the field which is formed by two overlapping fields) is shown as a field 16B, and a triple overlaid coil field (i.e., the field which is formed by three overlapping fields) is shown as field 16C.

The first coil 14A may be configured to be adhered to a patient body part, e.g., a patient's head, containing the patient's brain, and configured to emit a first magnetic field. Similarly, the second coil 14B may be configured to be adhered to the patient body part, simultaneously with the first coil 14A, and to emit a second magnetic field. Further, the third coil 14C may be configured to be adhered to the patient body part, simultaneously with the first coil 14A and the second coil 14B, and to emit a third magnetic field. In some aspects, the first magnetic field, the second magnetic field and/or the third magnetic field overlap within the patient body part. Furthermore, as shown in FIGS. 1 and 2, the first coil 14A, the second coil 14B, and the third coil 14C may be adhered to different portions of the patient body part, to provide enhanced and/or more precisely targeted TMS to the patient 11.

It is known that TMS coils associated with conventional TMS devices have several deficiencies. Further, localized discomfort is a leading adverse treatment effect of conventional TMS devices. As localized discomfort is expected to be experienced from the conventional TMS devices, a proportion of recipients/patients may either forgo the treatment or suspend treatment prematurely. Furthermore, it is known that the magnetic fields generated by TMS coils diminish with increasing distance from the coil, requiring higher amplitude electrical currents to induce electric current in brain tissue further from the skin surface. This makes it difficult to employ TMS coils to treat tissue regions more than 6 centimeters from the scalp surface. The conventional TMS devices target such tissue regions by employing higher amplitude TMS. A disadvantage to this approach includes involvement of more non-target tissues.

It is known that conventional TMS devices employ a single coil of varying designs to achieve the desired diagnostic or therapeutic effect. Some of the known coil designs include round coils, figure eight (or “butterfly”) coils, four leafed coils, double-cone coils, Hesed (H-core) circular crown coils, etc. It is hypothesized that disadvantages associated with the conventional TMS devices stem from this “single” coil design.

The system 10 address the challenges associated with the conventional TMS devices by employing two or more TMS coils (e.g., three coils 14A, 14B, 14C). The coils may be configured such that the magnetic fields extend in space to a common area of overlap coinciding with the tissue target of interest. The system 10 may enable greater magnetic field strength than experienced at any other more superficial site. The system 10 achieves a plurality of objectives, e.g., it reduces the potential for involvement of other non-target tissues or ferromagnetic materials, including tissues/materials which are more superficial on the orthogonal plane from the coil to the target site. The system 10 further offers better conceal treatment versus sham or placebo TMS, and enables more powerful, or deeper penetrating magnetic field, particularly to depths exceeding the present maximum of 6 cm. The system 10 further reduces the demand, and maintenance needs, of TMS coils by exerting less pronounced electrical currents on any given coil.

In some aspects, the system 10 may be employed for any diagnosis or disease in which TMS may become useful.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

1. A system for administering transcranial magnetic stimulation (TMS), comprising:
a first coil configured to be adhered to a patient body part, wherein the first coil emits a first magnetic field; and
a second coil configured to be adhered to the patient body part simultaneously with the first coil, wherein the second coil emits a second magnetic field, and wherein when the first coil and the second coil are adhered to the patient body part, the first magnetic field and the second magnetic field overlap within the patient body part.

2. The system of claim 1, wherein the patient body part is a head containing a brain.

3. The system of claim 1, wherein the first coil and the second coil are adhered to different portions of the patient body part.

4. The system of claim 1, further comprising a third coil configured to be adhered to the patient body part simultaneously with the first coil and the second coil, wherein the third coil emits a third magnetic field, and wherein the first magnetic field, the second magnetic field and the third magnetic field overlap within the patient body part.

5. The system of claim 4, wherein the first coil, the second coil and the third coil are adhered to different portions of the patient body part.

6. A method of administering transcranial magnetic stimulation, comprising:
providing the system of claim 1;
adhering the first coil and the second coil to the patient body part at preselected locations operative to produce a magnetic field overlap at a selected region of the patient body part; and
activating the first coil and the second coil at a selected strength for a selected time.
